(19) United States
(12) Patent Application Publication
(10) Pub. No.: US 2009/0088700 A1
(43) Pub. Date: Apr. 2, 2009

(54) APPARATUS AND METHOD FOR LIQUID SPRAY ADMINISTRATION

(75) Inventor: Hiroyuki Imbayashi, Hachioji-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/237,081

(22) Filed: Sep. 24, 2008

(30) Foreign Application Priority Data
Sep. 27, 2007 (JP) 2007-252048
Sep. 27, 2007 (JP) 2007-252049

(51) Int. Cl. A61M 5/00; A61M 31/00

(52) U.S. Cl. 604/181; 604/65

(57) ABSTRACT

A liquid spray administration apparatus which is inserted into a body to spray and administer liquid, including a liquid filling unit which is filled with the liquid, a catheter which is connected to the liquid filling unit at one end while having a spray nozzle at the other end, the spray nozzle spraying the liquid, a voltage generation unit which generates a voltage, and a voltage application unit which is disposed near the liquid filling unit or one end of the catheter, and applies the voltage generated from the voltage generation unit to the liquid in the spray nozzle through the liquid in the catheter, wherein the liquid is applied is sprayed and administered in a form of a charged misty liquid fine particulate from the spray nozzle toward a region, the region having a potential which is different from that of the liquid in the spray nozzle.

### CROSS-REFERENCE TO RELATED APPLICATIONS

This application is based upon and claims the benefit of priority from prior Japanese Patent Applications No. 2007-252048, filed Sep. 27, 2007; and No. 2007-252049, filed Sep. 27, 2007, the entire contents of both of which are incorporated herein by reference.

### BACKGROUND OF THE INVENTION

1. Field of the Invention

The present invention relates to a liquid spray administration apparatus and a liquid spray administration method for spraying and administering is liquid (for example, medical solution) including a medical agent to a target region in a subject.

2. Description of the Related Art

Recently, in order to efficiently administer a liquid containing a therapeutic medical solution to a desired region in a living body such as a patient or an animal which are subjects, it is considered that desirably the medical solution is administered as close to the region (affected area) as possible. For a nucleic-acid medicine which is being developed as a new medicine, it is considered preferable for the nucleic acid medicine to be brought close to the affected area and locally administered to the affected area from the viewpoint of efficiency deterioration due to metabolic decomposition. Thus, there is a need for being able to securely administer the necessary amount of the medical solution to the affected region to lower the side-effects experienced by the patient.

For example, a medical solution administration method disclosed in Jpn. PCT National Publication No. 2006-527023 will be described below with reference to FIG. 10.

A catheter 120 and a catheter assembly 110 are disclosed in the medical solution administration method. For example, the catheter 120 and the catheter assembly 110 deliver a remedy (hereinafter referred to as fine-particulate remedy) which is a fine-particulate medical solution to a heart, which is a target region in the body. The delivered fine-particulate remedy includes an aerosol type fine-particulate remedy and a dry powder type fine-particulate remedy. Each of the two types of fine-particulate remedies is biased by a biasing mechanism which generates a supersonic flow, in which the fine-particulate remedy is caused to make a transition from a stationary state in the catheter 120 to a state of being moved toward the target region, whereby the fine-particulate remedy is delivered to the heart. The biasing mechanism biases the fine-particulate remedy using a pressurized gas, vacuum, a centripetal force, a plunger, or an electric potential gradient, and the like.

Since a remedy having a high solid/fluid ratio does not easily pass through a delivery lumen, it is necessary to utilize a solvent in the remedy in order to realize a practical solid/fluid ratio. However, in such cases, unfortunately, the solvent may be harmful to the target region and may not be compatible with the remedy.

Therefore, Jpn. PCT National Publication No. 2006-527023 provides a delivery apparatus and method for efficiently delivering a remedy which easily passes through the delivery lumen to the target region.

In the delivery apparatus, when an electric potential gradient is used in the biasing mechanism, the catheter assembly 110 includes the catheter 120. The catheter 120 includes a cavity, a proximal end 130, and a distal end 135. The cavity is extended over the total length of the catheter 120, the proximal end 130 includes an active electrode, and the distal end 135 includes a counter electrode and a nozzle 180.

The catheter assembly 110 includes a battery or a pulse generator, which is an electric energy source, and the active electrode and the counter electrode are connected to the electric energy source. The charged remedy is delivered through the active electrode, moved along the electric potential gradient formed by a circuit including the active electrode and the counter electrode, and reaches the target region through the nozzle 180 of the catheter 120. Jpn. PCT National Publication No. 2006-527023 discloses that, in the case where the delivered remedy is positively charged, an anode becomes the active electrode while a cathode becomes the counter electrode, thereby completing an electric circuit. In the case where the delivered remedy is negatively charged, the cathode becomes the active electrode while the anode becomes the counter electrode.

### BRIEF SUMMARY OF THE INVENTION

The present invention provides a liquid spray administration apparatus and a liquid spray administration method in which a liquid is selectively administered to the target region in the living body without the use of gas pressure.

The present invention provides a liquid spray administration apparatus which is inserted into a body to spray and administer a liquid, comprising: a liquid filling unit which is filled with the liquid; a catheter which is connected to the liquid filling unit at one end while having a spray nozzle at the other end, the spray nozzle spraying the liquid; a voltage generation unit which generates a voltage; and a voltage application unit which is disposed near the liquid filling unit or one end of the catheter, and applies the voltage generated from the voltage generation unit to the liquid in the spray nozzle through the liquid in the catheter, wherein the liquid to which the voltage is applied is sprayed and administered in a form of a charged misty liquid fine particulate from the spray nozzle toward a region, the region having a potential which is different from that of the liquid in the spray nozzle.

The present invention provides a liquid spray administration apparatus which is inserted into a body to spray and administer a liquid fine particulate, comprising: a production unit which produces the charged fine particles from the liquid to which a voltage is applied; and a spray nozzle which sprays the charged fine particles to a desired region in the body, wherein the fine particles are sprayed and administered from the spray nozzle toward a region, the region having a potential which is different from that of the liquid in the spray nozzle.

The present invention provides a liquid spray administration method performed by a liquid spray administration apparatus which is inserted into a body to spray and administer a liquid, the method comprising: a step of filling a liquid filling unit with the liquid; a step of connecting the liquid filling unit and one end of a catheter; a step of generating a voltage; a step of applying the voltage to the liquid at the liquid filling unit or one end; and a step of spraying and administering the liquid to which the voltage is applied in a form of a charged misty liquid fine particulate from the spray nozzle toward a region, the spray nozzle being provided at the other end of the catheter, the region having a potential which is different from that of the liquid in the spray nozzle.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view showing a liquid spray administration apparatus according to a first embodiment of the invention;

FIG. 2 shows a disposal syringe and a catheter in spraying a medical solution;

FIG. 3 shows configurations of the disposal syringe and catheter;

FIG. 4 is a schematic view showing the other end of a catheter according to a second embodiment, and shows a state in which a dose of the medical solution is increased in a catheter insertion direction;

FIG. 5 is a schematic view showing the other end of the catheter of the second embodiment, and shows a state in which the dose of the medical solution is increased in a catheter radial (circumferential) direction;

FIG. 6 is a schematic view showing a liquid spray administration apparatus according to a third embodiment in spraying the medical solution;

FIG. 7 is a schematic view showing the liquid spray administration apparatus when a catheter is inserted into a forceps channel of an endoscope;

FIG. 8 is a schematic view showing a liquid spray administration apparatus according to a fourth embodiment in spraying the medical solution, and shows a disposal syringe and a catheter when the medical solution is sprayed by air;

FIG. 9 is a schematic view showing the other end of a catheter according to a fifth embodiments and shows a state in which the dose of the medical solution is increased in the catheter insertion direction; and

FIG. 10 shows an example of a conventional catheter assembly.

### DETAILED DESCRIPTION OF THE INVENTION

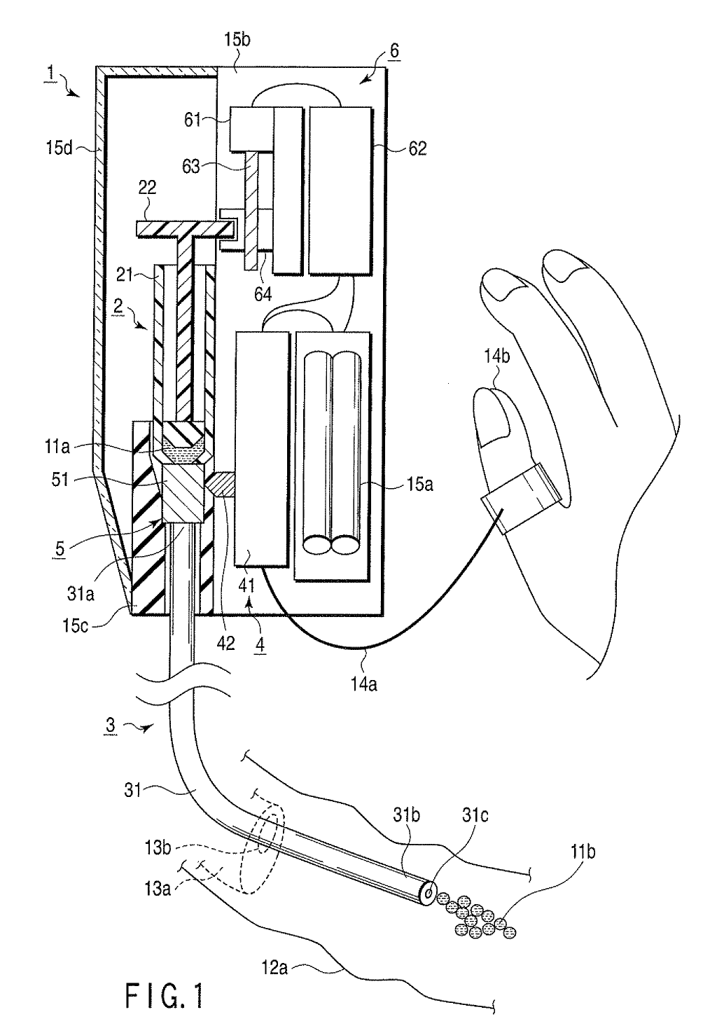

A first embodiment of the invention will be described with reference to FIGS. 1 to 3.

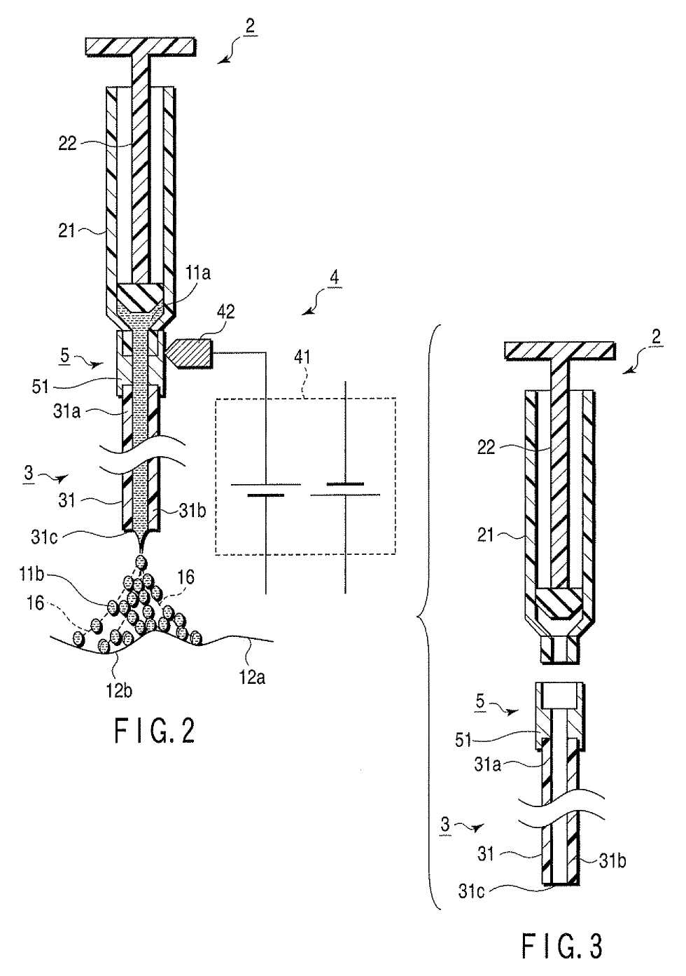

FIG. 1 is a schematic view showing a liquid spray administration apparatus according to the first embodiment of the invention. FIG. 2 shows a disposal syringe and a catheter in spraying a medical solution. FIG. 3 shows configurations of the disposal syringe and catheter.

Referring to FIG. 1, a liquid spray administration apparatus 1 mainly includes a liquid filling unit 2, a spray unit 3, a voltage generation unit 4, and a voltage application unit 5. The liquid filling unit (hereinafter referred to as medical solution filling unit) 2 is filled with a liquid (for example, a medical solution 11a). The spray unit 3 sprays the medical solution 11a with which the medical solution filling unit 2 is filled to a target region (for example, an affected area 12b shown in FIG. 2) which is a desired region in a body (for example, a living body 12a of a patient which is a subject). The voltage generation unit 4 generates an applied voltage. The voltage application unit 5 applies the applied voltage generated from the voltage generation unit 4 to the medical solution 11a in the medical solution filling unit 2 or spray unit 3. The liquid spray administration apparatus 1 also includes a liquid sending mechanism (hereinafter referred to as medical solution sending mechanism) 6 which sends the medical solution 11a, to which the applied voltage is applied, from the medical solution filling unit 2 to a spray nozzle 31c of the spray unit 3.

The medical solution filling unit 2 includes a disposal syringe 21 and a piston 22. The disposal syringe 21 is a medical solution tank filled with the medical solution 11a. The piston 22 sends the medical solution 11a with which the disposal syringe 21 is filled to a catheter 31 of the spray unit 3. The piston 22 is moved toward the side of the catheter 31 in a lengthwise axis direction of the disposal syringe 21 to send the medical solution 11a from the disposal syringe 21 to the catheter 31. The disposal syringe 21 and the piston 22 are made of a plastic such as polypropylene. A gasket (sealing portion) at a distal end of the piston 22 is made of an elastic material such as a thermoplastic elastomer. In the catheter 31 provided in the spray unit 3, one end 31a is connected to the disposal syringe 21, and the medical solution 11a with which the disposal syringe 21 is filled is sprayed (ejected, or discharged) from the other end 31b inserted into the living body 12a to the affected area 12b of the living body 12a. One end 31a is disposed outside the body (outside the living body 12a).

The catheter 31 includes a spray nozzle 31c at the other end 31b. The spray nozzle 31c sprays the medical solution 11a to the affected area 12b in the form of a medical-solution fine particulate 11b at the other end 31b. The catheter 31 is made of a non-conductive material such as Teflon (registered trademark), which is a polytetrafluoroethylene resin, and has good flexibility. For example, the catheter 31 is a thin tube having an external diameter of about 0.3 mm, an internal diameter of about 0.1 mm, and a length of about 2000 mm. The spray nozzle 31c has a diameter of about 0.1 mm. As shown in FIG. 1, the catheter 31 is inserted into a forceps channel 13b of an endoscope 13a, and the spray nozzle 31c is disposed so as to face the affected area 12b. At this point, the catheter 31 is advanced or retreated in a direction in which the catheter 31 is inserted, thereby bringing the spray nozzle 31c close to or taking the spray nozzle 31c away from the affected area 12b.

The voltage generation unit 4 includes a voltage generation circuit 41 and an electrode contact member 42.

The voltage generation circuit 41 generates a high voltage, which is an applied voltage of 1 kV or more, from a power supply such as a battery 15a, and supplies this high voltage to the voltage application unit 5 through the electrode contact member 42. An operation start switch (not shown) controls an operation of the voltage generation circuit 41 to adjust the generation of the high voltage. As shown in FIG. 2, the voltage generation circuit 41 selects the polarity of the high voltage, that is, either positive-side polarity or negative-side polarity.

The electrode contact member 42 has a needle shape tapered toward an applied voltage direction in order to concentrate the application of the high voltage in the voltage application unit 5. For example, a stainless steel electrode is suitable as the electrode contact member 42. A gold-plated contact probe of a general-purpose electric contactor may be used as the electrode contact member 42.

A ground band 14a is extracted from the voltage generation circuit 41 to the outside of the liquid spray administration apparatus 1. The ground band 14a having a potential of 0 V contacts part of the living body 12a outside the liquid spray administration apparatus 1. In the first embodiment, for example, the part of the living body 12a is a finger 14b, as shown in FIG. 1. Therefore, the affected area 12b of the living body 12a is grounded. That is, the affected area 12b has a potential different from that of the medical solution 11a of the spray nozzle 31c, to which the high voltage is applied. That is, the affected area 12b is the region having a potential which is different from that of the medical solution 11a in the spray nozzle 31c.

A high-resistance circuit (not shown) or an overcurrent detection circuit (not shown) is incorporated in the voltage generation circuit 41 as a safety countermeasure against the high voltage. A protective high resistances which is the high-resistance circuit, is disposed in series in the electrode contact member 42 to prevent a spark or an electric shock affecting the living body 12a. The overcurrent detection circuit detects the current which is passed through the voltage generation circuit 41 when the voltage generation circuit 41 supplies the high voltage to the voltage application unit 5. When the current is not lower than a predetermined value, the overcurrent detection circuit stops the voltage generation circuit 41 to stop the generation of the high voltage. From the viewpoint of safety of the living body 12a, preferably a setting value of the overcurrent detection circuit is not more than about 100 μA or at least about 10 μA.

Other safety countermeasures against high voltages are also provided in the voltage generation circuit 41. For example, the voltage generation circuit 41 works with the medical solution sending mechanism 6 to stop the generation of high voltages when the medical solution 11a runs out of the medical solution 11a in the disposal syringe 21 (for example, when the piston 22 is moved to the side of the catheter 31). In other words, the voltage generation circuit 41 generates a high voltage to supply the high voltage only when the medical solution sending mechanism 6 sends the medical solution 11a.

A transformer (not shown) may be provided in the voltage generation circuit 41 to boost the power supply, such as the battery 15a, to a high voltage.

A storage unit (not shown) may be provided in the voltage generation circuit 41. A table retaining a predetermined combination of the polarity, magnitude, and an application duration time of the applied voltage is stored in the storage unit. The application duration time is a period during which a high voltage is supplied to the voltage application unit 5 to cause the voltage application unit 5 to apply a high voltage to the medical solution 11a. When applying a high voltage to the voltage application unit 5, the voltage generation circuit 41 calls the table according to use application of the medical solution 11a, and the voltage generation circuit 41 may regulate at least one of the polarity, magnitude, and application duration time of the applied voltage corresponding to the use application.

A control terminal (not shown) connected to an external switch (not shown) may be incorporated in the voltage generation circuit 41. For example, a user manipulates the external switch which is a dial, whereby the voltage generation circuit 41 may variably set and control at least one of the polarity, magnitude, and application duration time of the applied voltage.

A diameter of the medical-solution fine particulate 11b is changed when the voltage generation circuit 41 varies the magnitude of the applied voltage.

The voltage application unit 5 includes a voltage application electrode 51.

The voltage application electrode 51 applies the high voltage supplied from the voltage generation circuit 41 through the electrode contact member 42 to the medical solution 11a in the catheter 31. Particularly, the voltage application electrode 51 applies the high voltage to the medical solution 11a of the spray nozzle 31c through the medical solution 11a of the catheter 31. This enables the medical solution 11a to be charged and sprayed from the spray nozzle 31c in the form of a misty fine-particulate (medical-solution fine particulate 11b). At this point, the medical-solution fine particulate 11b is charged in the positive-side polarity when the high voltage has a positive-side polarity, and the medical-solution fine particulate 11b is charged in the negative-side polarity when the high voltage has a negative-side polarity. Generally the living body 12a has a potential near 0 V, and becomes 0 V by the ground band 14a in the first embodiment. Therefore, the living body 12a differs from the medical solution 11a (charged medical-solution fine particulate 11b) of the spray nozzle 31c in the potential. Accordingly, the medical-solution fine particulate 11b charged in the positive-side polarity or negative-side polarity adheres actively to the affected area 12b of the living body 12a having a potential which is different from that of the medical solution 11a of the spray nozzle 31c.

The voltage application electrode 51 having a cylindrical shape is made of a conductive metal, a conductive resin, or a resin in which a conductive film is formed. As shown in FIGS. 2 and 3, since the voltage application electrode 51 is strongly bonded to one end 31a using a bonding agent (not shown), the voltage application electrode 51 is disposed outside the living body 12a. An internal diameter of the voltage application electrode 51 is substantially equal to an internal diameter of the catheter 31, and the medical solution 11a contacts the internal diameter portion of the voltage application electrode 51. The medical solution 11a in the catheter 31 contacts the internal diameter portion of the voltage application electrode 51, whereby the voltage application electrode 51 applies the high voltage to the medical solution 11a.

The voltage application electrode 51 is detachably attached to the electrode contact member 42. The voltage application electrode 51 and the disposal syringe 21 are tightened in a screw-in manner, and are detachably attached to each other. That is, a constituent including the disposal syringe 21 and piston 22 and a constituent including the catheter 31 and voltage application electrode 51 are detachably attached to each other and disposed of as a disposable component after use, according to circumstances.

As described above, the disposal syringe 21 is filled with the medical solution 11a, the catheter 31 is connected to the disposal syringe 21 at one end 31a thereof, the voltage generation circuit 41 of the voltage generation unit 4 generates a high voltage, and the voltage application electrode 51 of the voltage application unit 5 applies the high voltage to the medical solution 11a. Therefore, the high-voltage-applied medical solution 11a is sprayed in the charged medical-solution fine particulate 11b. Thus, the disposal syringe 21, the catheter 31, the voltage generation unit 4, and the voltage application unit 5 constitute a producing unit which produces the charged medical-solution fine particulate 11b from the high-voltage-applied medical solution 11a.

The medical solution sending mechanism 6 includes a motor 61, a control circuit 62, a ball screw 63, and a movable unit 64. The motor 61 is a drive unit. The control circuit 62 controls the motor 61. The ball screw 63 is connected to the motor 61, and rotational motion of the motor 61 is transmitted to the ball screw 63. The movable unit 64 engages the ball screw 63, and is detachably attached to the piston 22. The movable unit 64 is moved along the ball screw 63 by transmitting the rotational motion of the motor 61 from the ball screw 63, thereby moving the piston 22 in the lengthwise axis direction of the disposal syringe 21.

The control circuit 62 controls the number of revolutions of the motor 61 to adjust a solution sending amount of the medical solution 11a which is sent from the disposal syringe 21 to the spray nozzle 31c, and the control circuit 62 controls a rotational speed of the motor 61 to adjust a solution sending speed of the medical solution 11a which is sent from the disposal syringe 21 to the spray nozzle 31c. The motor 61 is rotated based on an instruction issued from the control circuit 62, and moves the movable unit 64 through the ball screw 63 to move the piston 22 toward the side of the catheter 31 along the lengthwise axis direction of the disposal syringe 21, thereby sending the medical solution 11a from the disposal syringe 21 to the spray nozzle 31c. The voltage application electrode 51 applies the voltage to the medical solution 11a. In detail, the medical solution 11a is sent from the disposal syringe 21 to one end 31a, the voltage application electrode 51 applies the voltage to the medical solution 11a, and the medical solution 11a is sent from one end 31a to the spray nozzle 31c.

The control circuit 62 may include a storage unit (not shown). A table retaining a predetermined combination of the solution sending amount (the number of revolutions of the motor 61) and solution sending speed (rotational speed of the motor 61) of the medical solution 11a, which is sent from the disposal syringe 21 to the spray nozzle 31c, is stored in the storage unit. In sending the medical solution 11a, the control circuit 62 calls the table according to the use application of the medical solution 11a, and may regulate (control) at least one of the solution sending amount and solution sending speed corresponding to the use application.

A control terminal (not shown) connected to an external switch (not shown) may be incorporated in the control circuit 62. For example, a user manipulates the external switch which is a dial, whereby the control circuit 62 may variably set and control at least one of the solution sending amount (the number of revolutions of the motor 61) and solution sending speed (rotational speed of the motor 61) of the medical solution 11a.

A diameter of the medical-solution fine particulate 11b is changed when the control circuit 62 varies the solution sending speed.

The battery 15a supplies an electric power to the medical solution sending mechanism 6 and the voltage generation circuit 41.

The medical solution sending mechanism 6, the voltage generation circuit 41, the electrode contact member 42, and the battery 15a are incorporated in a case 15b. The disposal syringe 21 filled with the medical solution 11a, the piston 22, the voltage application electrode 51, and the catheter 31 are connected to one another, and are inserted into a disposal syringe fixing frame 15c which is an electrically insulating portion such as rubber. The piston 22 is connected to the movable unit 64 to form the liquid spray administration apparatus 1.

Because the voltage application electrode 51 is connected to the electrode contact member 42 in the disposal syringe fixing frame 15c, the disposal syringe fixing frame 15c and the case 15b prevent the voltage application electrode 51 and the electrode contact member 42 from contacting anything present externally.

An openable transparent resin cover 15d, which prevents a finger 14b from being caught, is disposed in a portion in which the piston 22 is moved.

The disposal syringe 21, the piston 22, the catheter 31, and the voltage application electrode 51 are detachably attached to the movable unit 64 of the medical solution sending mechanism 6 and the electrode contact member 42 of the voltage generation unit 4, and are disposed of as the disposable component after use, according to circumstances.

Therefore, as described above, in disposing of the disposal syringe 21, the piston 22, the catheter 31, and the voltage application electrode 51 after use, the voltage generation unit 4, the medical solution sending mechanism 6, the battery 15a, the case 15b, the disposal syringe fixing frame 15c, and the cover 15d can be connected to an unused disposal syringe 21, piston 22, catheter 31, and voltage application electrode 51 and reused as a reuse component.

Obviously, an operator can manually push the piston 22 without the use of the medical solution sending mechanism 6 to send the medical solution Ha from the disposal syringe 21 to the spray nozzle 31c.

An operation method in the first embodiment will be described below.

The disposal syringe 21 and the voltage application electrode 51 are tightened in a screw-in manner, and are integral with the catheter 31.

The disposal syringe 21 is filled with the medical solution 11a having a desired amount, and the piston 22 is disposed in the disposal syringe 21. The desired amount shall mean a proper amount of which the medical solution 11a is used to remedy the affected area 12b and an amount which is sent from the disposal syringe 21 to the spray nozzle 31c by the medical solution sending mechanism 6.

After being connected to one another, the disposal syringe 21, the voltage application electrode 51, and the catheter 31 are inserted into the disposal syringe fixing frame 15c. The piston 22 is connected to the movable unit 64 disposed in the case 15b, and the voltage application electrode 51 is connected to the electrode contact member 42. The cover 15d is closed. Therefore, the liquid spray administration apparatus 1 is formed.

The catheter 31 is inserted into the forceps channel 13b of the endoscope 13a. While the affected area 12b is observed by an observation optical system (not shown) provided in the endoscope 13a, the catheter 31 is disposed such that the spray nozzle 31c faces the affected area 12b.

In the liquid spray administration apparatus 1, when a user uses an operation start switch (not shown) to provide an instruction for starting the operation, the control circuit 62 causes the motor 61 to be rotated. Therefore, the piston 22 is moved toward the side of the catheter 31 in the lengthwise axis direction of the disposal syringe 21 to push the appropriate amount of medical solution 11a in the disposal syringe 21 into the catheter 31. The medical solution 11a of the proper amount is sent from the disposal syringe 21 to the spray nozzle 31c. At this point, the high voltage generated from the voltage generation circuit 41 is supplied to the voltage application electrode 51 through the electrode contact member 42. The voltage application electrode 51 applies the high voltage to the medical solution 11a in the catheter 31. That is, the high voltage is applied to the medical solution 11a in the catheter 31.

As shown in FIG. 2, the high-voltage-applied medical solution 11a in the spray nozzle 31c is sprayed from the spray nozzle 31c in the form of the charged misty medical-solution fine particulate 11b.

Particularly, in the medical solution 11a in the spray nozzle 31c, a boundary of a liquid and gas is formed between the medical solution 11a and the outside air. When the high voltage acts on the boundary, the boundary becomes electrohydrodynamically unstable to generate an unstable point by electrostatic force acting on a surface of the medical solution 11a. The charged misty medical-solution fine particulate 11b is sprayed from the unstable point. When the charge density at the boundary in the spray nozzle 31c reaches a critical value because the high voltage acts on the boundary, a thin liquid thread is drawn from the surface of the medical solution 11a, and the liquid thread is expanded and contracted. At this point, the medical solution 11a is dissociated into a fine particulate 11b from the distal end of the liquid thread. When the high voltage is further increased, the boundary in the thin thread becomes further unstable, generating many unstable points. The medical solution 11a is sprayed in the form of the charged misty medical-solution fine particulate 11b from the many unstable points in the spray nozzle 31c.

Because the catheter 31 is made of a non-conductive material, such as polytetrafluoroethylene resin, the medical solution 11a is sprayed without remaining in the catheter 31.

As described above, in applying the high voltage to the medical solution 11a, a potential difference is generated between the living body 12a and the medical solution 11a in the spray nozzle 31c. That is, the voltage generation circuit 41 generates a high voltage and applies the high voltage to the medical solution 11a, generating the potential difference between the living body 12a and the medical solution 11a in the spray nozzle 31c. At this point, an electrical flux line 16 is formed from the spray nozzle 31c toward the living body 12a as shown in FIG. 2. The charged misty medical-solution fine particulate 11b follows the electrical flux line 16 from the spray nozzle 31c, and is selectively administered toward the living body 12a within a range in which the electrical flux line 16 is formed. The living body 12a has a potential different from that of the medical solution 11a in the spray nozzle 31c. That is, the affected area 12b is the region having a potential which is different from that of the medical solution 11a in the spray nozzle 31c. At this point, the charged medical-solution fine particulate 11b adheres to the living body 12a having a potential of 0 V. That is, the medical solution 11a is selectively administered in the form of a medical-solution fine particulate 11b only to the affected area 12b in the living body 12a, which has a potential different from that of the medical solution 11a in the spray nozzle 31c. The charged medical-solution fine particulate 11b adheres securely to the living body 12a having a potential of 0 V. Thus, in the liquid spray administration apparatus 1, the high-voltage-applied medical solution ha in the spray nozzle 31c is sprayed and administered from the spray nozzle 31c in the form of a charged misty medical-solution fine particulate 11b as shown in FIG. 2. At this point, in the liquid spray administration apparatus 1, the medical-solution fine particulate 11b follows the electrical flux line 16, and is selectively sprayed and administered toward the living body 12a within the range in which the electrical flux line 16 is formed. The living body 12a has a potential different from that of the medical solution 11a in the spray nozzle 31c. That is, the affected area 12b is the region having a potential which is different from that of the medical solution 11a in the spray nozzle 31c.

Thus, in the first embodiment, the medical solution 11a can selectively be administered to the affected area 12b in the living body 12a without the use of gas pressure. Additionally, because the medical-solution fine particulate 11b is charged, the medical-solution fine particulate 11b can securely adhere to the affected area 12b without flowing out from the affected area 12b. Therefore, in the first embodiment, the medical solution 11a can be administered solely to the target region requiring the medical solution 11a, and the medical solution 11a of the proper amount can be administered to improve the therapeutic effect.

In the first embodiment, the medical solution 11a is not misted in the disposal syringe 21 and the catheter 31, the medical solution 11a in the misted state is not sent to the spray nozzle 31c, and the medical solution 11a is administered to the affected area 12b without the use of gas pressure. Further, in the first embodiment, the catheter 31 is filled with the medical solution 11a, which is misted at the spray nozzle 31c, and administered to the affected area 12b according to the electrical flux line 16. Therefore, the medical solution 11a can be prevented from remaining in the catheter 31, and a drop in dosage of the medical solution 11a to the affected area 12b due to diffusion by gas pressure can be prevented. In the first embodiment, adhesion of the medical solution 11a dripping off from the spray nozzle 31c to regions other than the affected area 12b can be prevented, so that the medical solution 11a can selectively be administered to the affected area 12b in the living body 12a. Additionally, a medical solution 11a of the proper amount can be administered to the affected area 12b in the living body 12a.

Sometimes, a bubble medical solution or a medical solution containing dispersed particles is used as the medical solution 11a. For example, in an ultrasonic misting principle utilized in a nebulizer, because the bubble medical solution 11a obstructs propagation of an ultrasound wave, the misting is frequently stopped. According to the theory behind ultrasonic misting, a mesh having a hole diameter of several micrometers is used in order to obtain a fine medical-solution fine particulate 11b. However, in misting the medical solution 11a containing dispersed particles, the misting is stopped when the dispersed particles clog up the mesh. However, in the first embodiment, for the bubble medical solution or the medical solution containing dispersed particles, neither ultrasound or a mesh is used, but a high voltage is used. Therefore, the bubble medical solution 11a or the medical solution 11a containing dispersed particles can easily be sprayed in the form of the charged completely-misty medical-solution fine particulate 11b, the medical solution 11a can selectively be administered to the affected area 12b in the living body 12a, and the medical solution 11a of the proper amount can be administered to the affected area 12b in the living body 12a.

As described above, the charged medical-solution fine particulate 11b follows the electrical flux line 16 from the spray nozzle 31c, and is selectively administered toward the living body 12a within the is range in which the electrical flux line 16 is formed. The living body 12a has a potential different from that of the medical solution 11a in the spray nozzle 31c. That is, the affected area 12b is the region having a potential which is different from that of the medical solution 11a in the spray nozzle 31c. At this point, the medical-solution fine particulate 11b adheres securely to the living body la having a potential of 0 V.

Therefore, the amount of the medical solution 11a which is sent from the disposal syringe 21 to the spray nozzle 31c by the medical solution sending mechanism 6 becomes substantially equal to the amount of the medical solution 11a (the medical-solution fine particulate 11b) which sprayed and administered from the spray nozzle 31c, and the amount of the medical solution 11a sent from the disposal syringe 21 to the spray nozzle 31c becomes substantially equal to the amount of the medical solution 11a adhering to the affected area 12b. In other words, the amount of the medical solution lie with which the disposal syringe 21 is initially filled (or the solution sending amount of the medical solution 11a from the disposal syringe 21 to the spray nozzle 31c) is substantially equal to the dose supplied to the affected area 12b.

Thus, in the first embodiment, a medical solution 11a of the proper amount can be administered to the affected area 12b in the living body 12a without the use of gas pressure. More particularly, in the first embodiment, a medical solution of the proper amount with which the disposal syringe 21 is filled can be administered to the affected area 12b in the living body 12a without decreasing the medical solution while the proper amount is maintained.

In the first embodiment, when the medical solution 11a has a conductivity of 1×10⁻¹⁰ (S/m) to 1×10⁻¹ (S/m), a diameter of the medical-solution fine particulate 11b can be changed by varying at least one of the solution sending speed of the medical solution 11a controlled by the medical solution sending mechanism 6 and the magnitude of the applied voltage controlled by the voltage generation circuit 41 of the voltage generation unit 4.

For example, in the first embodiment, a diameter of the medical-solution fine particulate 11b can be decreased by increasing the applied voltage.

For example, in the first embodiment, the medical solution sending mechanism 6 can enhance the solution sending speed to shorten the period during which the unstable point is generated. Therefore, a diameter of the medical-solution fine particulate 11b is increased because the medical-solution fine particulate 11b is sprayed before becoming the completely misted state. Accordingly, the solution sending speed can be enhanced to increase a diameter of the medical-solution fine particulate 11b, and the hole diameter of the spray nozzle 31c can be decreased to decrease a diameter of the medical-solution fine particulate 11b.

Therefore, the optimum diameter of the particles in the medical-solution fine particulate 11b can be selected according to the affected area 12b, and the medical-solution fine particulate 11b having particles of the optimum diameter can selectively be administered to the affected area 12b with the proper amount.

In the first embodiment, for example, the medical solution 11a is distilled water having a conductivity 1×10⁻⁶ (S/m), the spray nozzle 31c has a diameter of about 0.1 mm, the applied voltage is set to about +5 kV, and the solution sending speed is set to about 0.1 mL/min. In such cases, the particles of the medical-solution fine particulate 11b having a diameter of about 20 μm to about 60 μm can selectively be administered to the affected area 12b in the living body 12a with the proper amount.

In the first embodiment, for example, the medical solution 11a is distilled water having a conductivity 1×10⁻⁶ (S/m), the spray nozzle 31c has a diameter of about 0.05 mm, the applied voltage is set to about +10 kV, and the solution sending speed is set to about 0.05 mL/min. In such cases, the particles of the medical-solution fine particulate 11b of a diameter of about 2 μm to about 10 μm can selectively be administered to the affected area 12b in the living body 12a in the proper amount.

In the first embodiment, for example, the medical solution 11a is distilled water having a conductivity 1×10⁻⁶ (S/m), the spray nozzle 31c has a diameter of about 0.5 mm, the applied voltage is set to about +5 kV, and the solution sending speed is set to about 0.3 mL/mm. In such cases, the particles of medical-solution fine particulate 11b having a diameter of about 100 μm to about 300 μm can selectively be administered to the affected area 12b in the living body 12a in the proper amount.

In the first embodiment, the medical solution sending mechanism 6 enhances the solution sending speed, and the voltage generation circuit 41 of the voltage generation unit 4 increases the applied voltage, whereby the dose of the medical solution 11a per unit time can be increased while the diameter of the particles of the medical-solution fine particulate 11b is kept constant.

In the case where the medical-solution fine particulate 11b is not charged, a force for maintaining a spherical shape acts on the medical-solution fine particulate 11b due to the surface tension of the medical-solution fine particulate 11b. The force becomes stronger as the diameter of the particles of medical-solution fine particulate 11b is decreased. Therefore, when the medical-solution fine particulate 11b is not charged, there is easily generated a dry fog phenomenon in which the medical-solution fine particulate 11b does not adhere to the surface of the living body 12a, but flies out. When the medical solution 11a is administered, the dose is an item which should be managed correctly. Therefore, in the first embodiment, the medical-solution fine particulate 11b is charged, so that the medical-solution fine particulate 11b can actively adhere to the affected area 12b. Accordingly, the medical solution 11a can selectively be administered to the affected area 12b in the living body 12a, and the medical solution 11a of the proper amount can be administered to the affected area 12b in the living body 12a, and the dose of the medical solution 11a to the affected area 12b can be managed correctly.

In particular, in the case where the medical solution 11a is administered to a lung or alveoli of a respiratory system, the uncharged medical-solution fine particulate 11b is easily discharged from a mouth by exhalation. Therefore, in the nebulizer used in an inhalation therapy, a medical solution spraying operation in which the medical solution 11a is sprayed at a suction timing is required in conjunction with exhalation. However, in the first embodiment, because the medical-solution fine particulate 11b is charged, the medical solution 11a can adhere to the affected area 12b irrespective of the exhalation of breath. Additionally, because the medical-solution fine particulate 11b is charged, the spray nozzle 31c is brought close to the affected area 12b in the lung, which allows the medical solution 11a to be selectively administered to the affected area 12b irrespective of the exhalation of breath. Therefore, the medical solution 11a of the proper amount can be administered to the affected area 12b in the living body 12a.

In the first embodiment, the catheter 31 is inserted into the forceps channel 13b of the endoscope 13a, and is advanced and retreated along the insertion direction of the catheter 31, and the electrical flux line 16 can be formed while the affected area 12b is observed by the observation optical system. That is, while the affected area 12b is observed by the observation optical system, the medical solution 11a can selectively be administered to the affected area 12b in the living body 12a, and the medical solution 11a of the proper amount can be administered to the affected area 12b in the living body 12a.

In the first embodiment, the catheter 31 is advanced along the insertion direction of the catheter 31 to bring the spray nozzle 31c close to the living body 12a, so that the administration area of the affected area 12b can be decreased. Further, the catheter 31 is retreated along the insertion direction of the catheter 31 to take the spray nozzle 31c away from the living body 12a, so that the administration area of the affected area 12b can be increased. That is, even if the dose per unit area is increased or decreased, because the medical-solution fine particulate 11b is charged, the medical solution 11a can be administered to the affected area 12b, and the medical solution 11a of the proper amount can be administered to the affected area 12b in the living body 12a.

In the first embodiment, because the voltage generation circuit 41 changes the polarity of the applied voltage, the charged polarity of the medical-solution fine particulate 11b can be selected from the positive-side polarity and the negative-side polarity. The medical-solution fine particulate 11b charged in the positive-side polarity or negative-side polarity is selectively sprayed, so that the medical solution 11a of the proper amount can selectively be administered to the affected area 12b in the living body 12a. The disposal syringe 21 and the catheter 31 are detachably attached to each other. Therefore, in the case where at least two kinds of medical solutions 11a are administered, only the disposal syringe 21 filled with the medical solution 11a is exchanged while the catheter 31 is disposed in the living body 12a, and the polarity can be selected according to an electric characteristic (ionization characteristic in the solution) of the medical solution 11a.

In the first embodiment, the operation start switch can turn on and off the voltage generation circuit 41 to control the generation of the high voltage, and the spraying can instantaneously be started and stopped according to the presence or absence of the high voltage application. Therefore, the voltage generation circuit 41 sets the application duration time, and the presence or absence of high voltage application is controlled by turning on or off the voltage generation circuit 41, so that the medical-solution fine particulate 11b can selectively be administered to the affected area 12b for an appropriate time, and the medical-solution fine particulate 11b of the proper amount can be administered to the affected area 12b for an appropriate time. That is, in the first embodiment, the appropriate administration duration, that is, the dose can be managed.

When the applied voltage is applied to the medical solution 11a, because there is nowhere except for the spray nozzle 31c to run for the electric field, that is, the electric field can only propagate through the spray nozzle 31c, the medical-solution fine particulate 11b is sprayed only from the spray nozzle 31c. In other words, when the applied voltage is applied to the medical solution 11a and, at the same time, when the micro spray nozzle 31c is provided, the spraying is not affected by the material, length, and diameter (internal diameter and external diameter) of the catheter 31. Therefore, in the first embodiment, a catheter 31 of any shape can be used when the applied voltage is applied to the medical solution 11a and, at the same time, when the micro spray nozzle 31c is provided.

In the first embodiment, the voltage generation circuit 41 and the finger 14b are brought into contact with each other by the ground band 14a, the living body 12a is set at a potential of 0 V, and the living body 12a has a potential different from that of the medical solution 11a in the spray nozzle 31c. Therefore, the charged medical-solution fine particulate 11b adheres securely to the affected area 12b, so that the medical solution 11a can selectively and easily be administered to the affected area 12b in the living body 12a, and the medical solution 11a of the proper amount can easily be administered to the affected area 12b in the living body 12a. In the first embodiment, a positive-polar-side potential or negative-polar-side potential of the living body 12a can be prevented, due to the provision of the ground band 14a, which attracts charged particles of the medical-solution fine particulate 11b, which can improve the safety.

The voltage application electrode 51 is disposed outside the living body 12a, and the spray nozzle 31c is disposed in the living body 12a. Therefore, the voltage application electrode 51 can safely be taken away from the living body 12a. The catheter 31 is made of a non-conductive material. Therefore, the liquid spray administration apparatus 1 is safe because a voltage is not applied to the living body 12a.

An electrostatic spray nozzle used in general painting is made of metal, and the nozzle becomes the voltage application electrode by itself, and the voltage is applied to the spray nozzle. In the first embodiment, because the liquid spray administration apparatus 1 is applied to the living body 12a, the catheter 31 made of a non-conductive material is used instead of an electrostatic spray nozzle, and the voltage application electrode 51 and the spray nozzle 31c of the catheter 31 are disposed at different positions. However, the charged medical-solution fine particulate 11b can be sprayed from the spray nozzle 31c, the voltage application electrode 51 can be taken away from the living body 12a, and the liquid spray administration apparatus 1 is safe because a voltage is not applied to the living body 12a.

In the first embodiment, the electric field is used in the spraying of the medical solution from the spray nozzle 31c, and the electrical flux line 16 is used in the adhesion of the medical solution, so that the power consumption of the battery 15a necessary for the adhesion of the medical-solution fine particulate 11b can be lessened.

In the first embodiment, at least one of the polarity, magnitude, and application duration time of the applied voltage can be controlled by the voltage generation circuit 41 of the voltage generation unit 4, and at least one of the solution sending amount and solution sending speed of the medical solution 11a can be controlled by the control circuit 62 of the medical solution sending mechanism 6. Therefore, the diameter of the particles, dose, and administration duration time of the medical-solution fine particulate 11b can desirably changed while the sprayed state of the medical-solution fine particulate 11b is confirmed.

In the first embodiment, the voltage application electrode 51 is disposed in the disposal syringe fixing frame 15c, and the voltage generation circuit 41 and the electrode contact member 42 are disposed in the case 15b. Therefore, the liquid spray administration apparatus 1 is safe for the living body 12a, because there is no chance of electric shock through contact of the living body 12a with these components.

In the first embodiment, in disposing of the disposal syringe 21, the piston 22, the catheter 31, and the voltage application electrode 51 after use, the voltage generation unit 4, the medical solution sending mechanism 6, the battery 15a, the case 15b, the disposal syringe fixing frame 15c, and the cover 15d can be reused as reuse components. Therefore, the cost can be reduced for the liquid spray administration apparatus 1.

It is only necessary to dispose the voltage application electrode 51 outside the living body and provide a location where the voltage application electrode 51 can contact the medical solution 11a. For example, the voltage application electrode 51 may be disposed near the disposal syringe 21 (medical solution filling unit 2), or one end 31a.

A second embodiment of the invention will be described below with reference to FIG. 4. The same components as the first embodiments are designated by the same numerals, and the descriptions thereof are omitted. FIG. 4 is a schematic view showing the other end of the catheter of the second embodiment.

In the second embodiment, as shown in FIG. 4, the catheter 31 has plural spray nozzles 31c in a distal-end surface 31d at the other end 31b. Because configurations other than the spray nozzle 31c are substantially similar to those of the first embodiment, detailed descriptions thereof are omitted.

Therefore, in the second embodiment, the dose of the medical solution 11a can be increased in the insertion direction of the catheter 31.

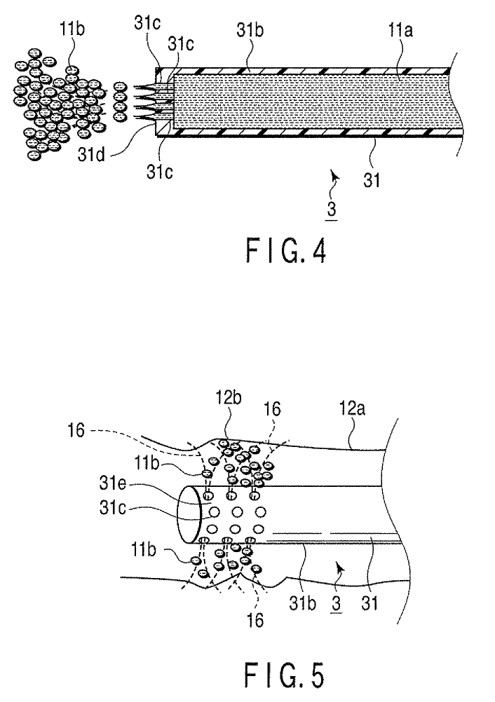

In the second embodiment, the configuration for increasing the dose of the medical solution 11a is not limited to the configuration of FIG. 4. For example, as shown in FIG. 5, the catheter 31 may have plural spray nozzles 31c in an outer circumferential surface 31e at the other end 31b.

Therefore, in the second embodiment, the dose of the medical solution 11a can be increased in the radial (circumferential) direction of the catheter 31.

In the second embodiment, as shown in FIGS. 4 and 5, because the number of spray nozzles 31c and the layout of the spray nozzles 31c can arbitrarily be set in the catheter 31, the dose of the medical solution 11a can be increased. In the second embodiment, similarly to the first embodiment, the electrical flux line 16 is formed between each spray nozzle 31c and the living body 12a, so that the medical-solution fine particulate 11b can be sprayed from each spray nozzle 31c. The increased number of spray nozzles 31c increases the dose, so that the medical solution 11a of the desired amount can selectively be administered in a short time, and the administration of the medical solution 11a of the proper amount can be completed in a short time.

In the case where the affected area 12b in the living body 12a is a disease affecting the whole circumference of a luminal portion in the living body 12a, in the second embodiment, at least one spray nozzle 31c is disposed in an outer circumferential surface 31e, so that the medical solution 11a can selectively be administered to the disease-affected part over the whole circumference of the luminal portion, and the medical solution 11a of the proper amount can be administered. In the second embodiment, the spray nozzles 31c are disposed in the outer circumferential surface 31e, the medical solution 41a can simultaneously be administered to the whole inner circumferential surface in the lumen when the liquid spray administration apparatus 1 is applied to the disease-affected part over the whole circumference of the luminal portion in the living body 12a. The dispositions of the spray nozzles 31c in the outer circumferential surface 31e can be used in dyeing the surface of the luminal portion in the living body 12a or in administering a diagnostic agent for early detection of cancer in a short time.

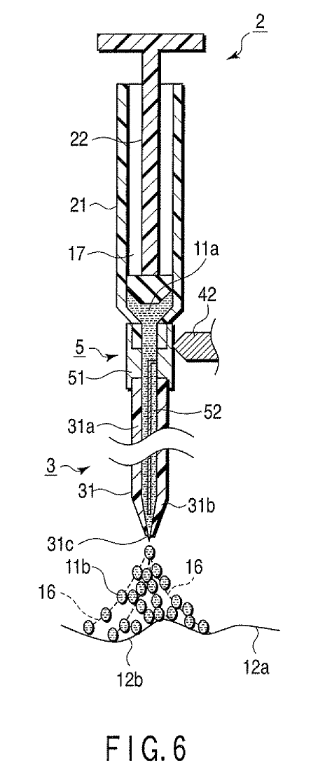

A third embodiment of the invention will be described below with reference to FIG. 6. The same components as in the first and second embodiments are designated by the same numerals, and the descriptions thereof are omitted. FIG. 6 is a schematic view showing a liquid spray administration apparatus of the third embodiment in spraying the medical solution. Because the voltage generation circuit 41, the medical solution sending mechanism 6, the battery 15a, the case 15b, the disposal syringe fixing frame 15c, and the cover 15d are similar to those of the first embodiment, these components are not shown in FIG. 6.

The voltage application electrode 51 of the third embodiment includes a voltage application thin wire 52. The voltage application thin wire 52 is connected to an inner circumferential portion (internal diameter portion) of the voltage application electrode 51, and is inserted into the catheter 31 to the neighborhood of the spray nozzle 31c. The voltage application thin wire 52 applies a high voltage to the medical solution 11a in the spray nozzle 31c through the medical solution 11a near the other end 31b in the catheter 31. The high voltage is supplied from the voltage generation circuit 41 through the electrode contact member 42 and the voltage application electrode 51. The material of the voltage application thin wire 52 preferably has a low electric resistance and stable characteristics for the medical solution 11a. More preferably, the voltage application thin wire 52 is made of platinum, gold, silver, or stainless steel on which a platinum thin film, a gold thin film, or a silver thin film is formed.

For example, in the case where the catheter 31 has an internal diameter of about 0.3 mm and a length of about 2000 mm, the voltage application thin wire 52 has a diameter of about 0.1 mm and a length of about 1980 mm.

The other end 31b of the catheter 31 is tapered by thermoforming such that the external diameter is decreased toward the spray nozzle 31c. A diameter of the spray nozzle 31c can be formed smaller than the internal diameter of about 0.3 mm or about 0.1 mm of the catheter 31. For example, the spray nozzle 31c has a diameter of about 70 μm.

The diameter of the voltage application thin wire 52 is larger than the diameter of the spray nozzle 31c, and the length of the voltage application thin wire 52 is shorter than the length of the catheter 31. Therefore, the voltage application thin wire 52 is not 5 exposed to the outside from the spray nozzle 31c. The length and diameter of the voltage application thin wire 52 are not limited to those in the third embodiment. It is only necessary that the voltage application thin wire 52 have the length or diameter which is not projected from the spray nozzle 31c and is extended to the neighborhood of the spray nozzle 31c.

Because other configurations are substantially similar to those of the first embodiment, detailed descriptions thereof are omitted.

In spraying the medical solution 11a, the voltage application thin wire 52 is extended to the neighborhood of the spray nozzle 31c, and a distance between the distal end of the voltage application thin wire 52 and the spray nozzle 31c becomes extremely short. A voltage is supplied to the voltage application thin wire 52 from the voltage generation circuit 41 through the electrode contact member 42 and the voltage application electrode 51, and the voltage is directly applied to the inside of the catheter 31 and the medical solution 11a in the spray nozzle 31c.

Because of the short distance between the voltage application thin wire 52 and the spray nozzle 31c, a larger potential difference between the medical solution 11a in the spray nozzle 31c and the living body 12a is generated than that in the first embodiment. At this point, a large electrical flux line 16 shown in FIG. 6 is formed from the spray nozzle 31c toward the living body 12a. Therefore, similarly to the first embodiment, the medical-solution fine particulate 11b sprayed from the spray nozzle 31c follows the large electrical flux line 16 shown in FIG. 6, and adheres strongly only to the living body 12a within the range in which the electrical flux line 16 is formed That is, the medical solution 11a of the proper amount is selectively administrated only to the affected area 12b in the living body 12a.

Thus, in the third embodiment, a large electrical flux line 16 is formed between the spray nozzle 31c and the surface of the living body 12a by the voltage application thin wire 52, so that the medical-solution fine particulate 11b can adhere strongly to the living body 12a, and the medical solution 11a of the proper amount can selectively be administrated to the affected area 12b in the living body 12a.

When a high voltage is applied to the medical solution 11a in the catheter 31 only in the internal diameter portion of the voltage application electrode 51, there is a risk of generating a voltage drop due to the length between the spray nozzle 31c and the internal diameter portion of the voltage application electrode 51. Therefore, there is a risk that a voltage which is lower than the high voltage supplied from the voltage generation circuit 41 will be applied to the medical solution 11a in the spray nozzle 31c. Accordingly, the voltage generation circuit 41 increases the voltage to allow for such voltage drop.

When the voltage application thin wire 52 is extended to the neighborhood of the spray nozzle 31c as in the third embodiment, because of the short distance between the voltage application thin wire 52 and the spray nozzle 31c, the generation of a voltage drop is prevented, and the high voltage generated from the voltage generation circuit 41 is directly applied to the medical solution 11a in the spray nozzle 31c. Therefore, it is not necessary for the voltage generation circuit 41 to generate an increased applied voltage to account for the voltage drop, so that the applied voltage necessary for the spraying can be lowered. That is, in the third embodiment, the high voltage generated from the voltage generation circuit 41 is directly applied to the medical solution 11a in the spray nozzle 31c through the voltage application thin wire 52, so that the applied voltage generated by the voltage generation circuit 41 can be lowered.

In the third embodiment, the high voltage generated from the voltage generation circuit 41 can be lowered as the distal end of the voltage application thin wire 52 is brought close to the spray nozzle 31c.

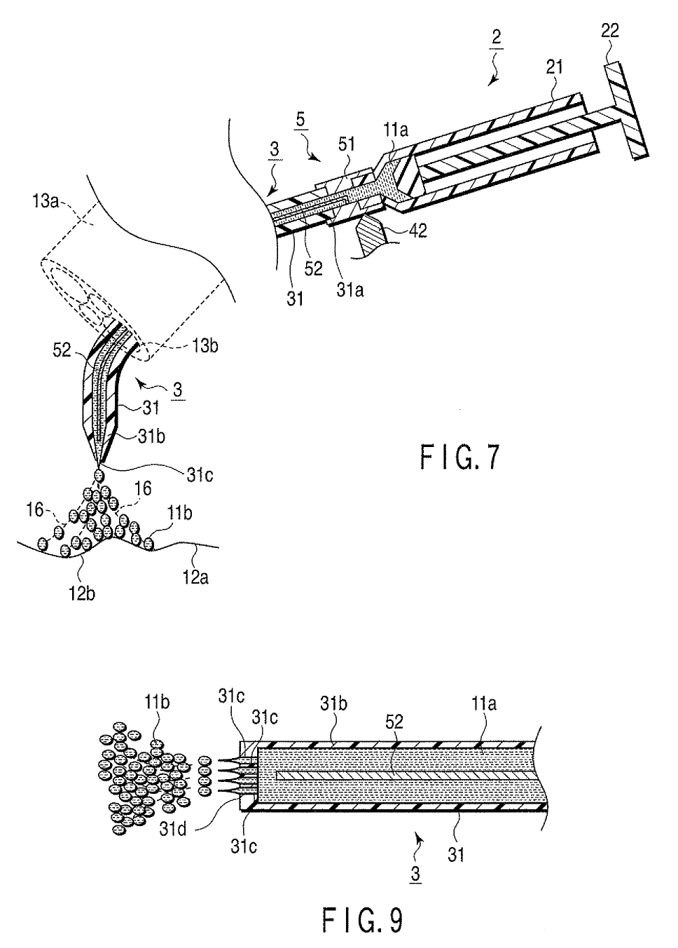

As shown in FIG. 7, because the catheter 31 is formed by a thin tube made of polytetrafluoroethylene resin, the catheter 31 is bent and buckled during use, the internal space of the catheter 31 becomes narrow, and a channel of the medical solution 11a is possibly blocked. However, in the third embodiment, the voltage application thin wire 52 is inserted into the catheter 31, so that the catheter 31 can be prevented from buckling.

In the third embodiment, the catheter 31 can be bent at any angle by plastically deforming the distal end of the catheter 31. The previously deformed plastic-deformed catheter 31 is inserted into the forceps channel 13b of the endoscope 13a, and the catheter 31 is returned to the previously-bent shape when the catheter 31 is projected from the forceps channel 13b of the endoscope 13a as shown in FIG. 7. Thus, the spraying direction can be set according to the use application by bending the distal end of the catheter 31. In the third embodiment, the medical solution 11a of the proper amount can selectively be administered to the affected area 12b in the living body 12a in this spraying direction.

In the third embodiment, the other end 31b of the catheter 31 is tapered by thermoforming, so that the spray nozzle 31c can be formed smaller. Therefore, the finer medical-solution fine particulate 11b can be sprayed by a smaller spray nozzle 31c.

In the third embodiment, because the other end 31b of the catheter 31 is tapered by thermoforming, the spray nozzle 31c can freely be formed and produced. Therefore, the production cost of the catheter 31 can be reduced.

In the third embodiment, the diameter of the spray nozzle 31c can be changed according to the diameter of the voltage application thin wire 52 as long as the diameter of the spray nozzle 31c is smaller than the diameter of the voltage application thin wire 52. Therefore, even if the voltage application thin wire 52 drops off from the voltage application electrode 51, the spray nozzle 31c prevents the voltage application thin wire 52 from being exposed to the outside, so that the liquid spray administration apparatus 1 can have safety.

In the third embodiment, the disposal syringe 21 need not be filled with air 17 like the first embodiment, or the disposal syringe 21 may be filled with the air 17 like the fourth embodiment.

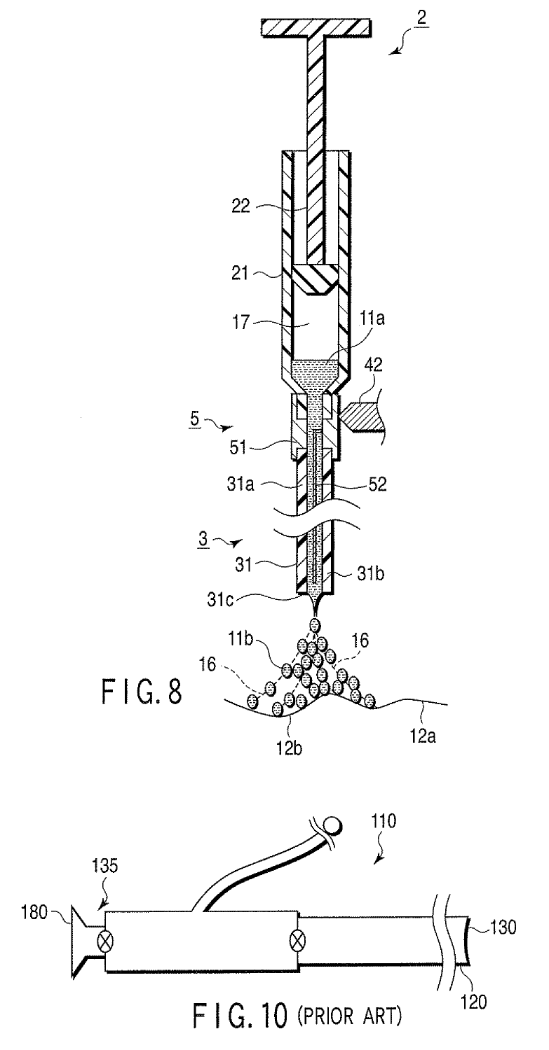

A fourth embodiment of the invention will be described below with reference to FIG. 8. The same components as the first to third embodiments are designated by the same numerals, and the descriptions thereof are omitted. FIG. 8 is a schematic view showing a liquid spray administration apparatus of the fourth embodiment in spraying a medical solution. Because the voltage generation circuit 41, the medical solution sending mechanism 6, the battery 15a, the case 15b, the disposal syringe fixing frame 15c, and the cover 15d are similar to those of the first embodiment, these components are not shown in FIG. 8.

In the fourth embodiment, the disposal syringe 21 is filled with the medical solution 11a of the proper amount and the air 17. As described above, the volume of the air 17 is not lower than the internal space of the catheter 31.

Similarly to the third embodiment, the voltage application electrode 51 of the fourth embodiment includes the voltage application thin wire 52.

Similarly to the first embodiment, for example, in the case where the catheter 31 has an external diameter of about 0.3 mm, internal diameter of about 0.1 mm, and length of about 2000 mm, the voltage application thin wire 52 has a diameter of about 0.03 mm and length of about 1980 mm. The length of the voltage application thin wire 52 is not limited to the fourth embodiment. It is only necessary that the voltage application thin wire 52 have the length which is not projected from the spray nozzle 31c and is extended to the neighborhood of the spray nozzle 31c.

Because other configurations are substantially similar to those of the first embodiment, the detailed descriptions thereof are omitted.

An operation method in the fourth embodiment will be described below.

The disposal syringe 21 is filled with the medical solution ha of the proper amount and a volume of air 17 which is not less than the internal space of the catheter 31, and the liquid spray administration apparatus 1 is formed like the first embodiment.

Similarly to the first embodiment, the piston 22 is moved to the side of the catheter 31 to push the air 17, thereby sending the medical solution 11a with which the disposal syringe 21 is initially filled from the disposal syringe 21 to the spray nozzle 31c. That is, the entire amount of medical solution 11a of the proper amount which should be administered is sent to the spray nozzle 31c without remaining in the catheter 31.

Similarly to the third embodiment, in spraying the medical solution 11a, the voltage application thin wire 52 is extended to the neighborhood of the spray nozzle 31c, and the distance between the distal end of the voltage application thin wire 52 and the spray nozzle 31c becomes extremely short. A voltage is supplied to the voltage application thin wire 52 from the voltage generation circuit 41 through the electrode contact member 42 and the voltage application electrode 51, and the voltage is directly applied to the inside of the catheter 31 and the medical solution 11a in the spray nozzle 31c.

Similarly to the third embodiment, because of the short distance between the voltage application thin wire 52 and the spray nozzle 31c, a larger potential difference between the medical solution 11a in the spray nozzle 31c and the living body 12a is generated than that of the first embodiment. At this point, a large electrical flux line 16 shown in FIG. 8 is formed from the spray nozzle 31c toward the living body 12a. Therefore, similarly to the first embodiment, the medical-solution fine particulate 11b sprayed from the spray nozzle 31c follows the large electrical flux line 16 shown in FIG. 8, and the medical-solution fine particulate 11b adheres strongly only to the living body 12a within the range in which the electrical flux line 16 is formed. That is, the medical solution 11a of the proper amount is administrated to the affected area 12b in the living body 12a.

Thus, in the fourth embodiment, a large electrical flux line 16 is formed between the spray nozzle 31c and the surface of the living body 12a by the voltage application thin wire 52, so that the medical-solution fine particulate 11b can adhere strongly to the living body 12a. Therefore, the medical-solution fine particulate 11b does not fly out to adhere to the surrounding portion of the affected area 12b, the amount of the medical solution 11a with which the disposal syringe 21 is initially filled (or the solution sending amount of the medical solution 11a from the disposal syringe 21 to the spray nozzle 31c) can be substantially equalized to the dose to be applied to the affected area 12b, and the medical solution 11a of the proper amount can be administered to the affected area 12b of the living body 12a.

In the fourth embodiment, similarly to the third embodiment, the high voltage generated from the voltage generation circuit 41 is directly applied to the medical solution 11a in the spray nozzle 31c through the voltage application thin wire 52, so that the applied voltage generated by the voltage generation circuit 41 can be lowered.

In the fourth embodiment, similarly to the third embodiment, the high voltage generated from the voltage generation circuit 41 can be lowered as the distal end of the voltage application thin wire 52 is brought close to the spray nozzle 31c.

Depending on the medical solution 11a, sometimes the air 17 within in the catheter 31 is expanded or gathered to form an air layer due to an external temperature. For example, in the catheter 31 in which the voltage application thin wire 52 is not disposed although only the voltage application electrode 51 is disposed like the first embodiment, when air is disposed between the voltage application electrode 51 and the spray nozzle 31c, the voltage may not be applied to the medical solution 11a in the spray nozzle 31c, which may interrupt the spraying.

However, in the fourth embodiment, the voltage application thin wire 52 is extended to the neighborhood of the spray nozzle 31c, so that interruption of the spraying can be prevented. Additionally, the voltage can surely be applied to the medical solution 11a in the spray nozzle 31c, and the medical solution 11a of the proper amount necessary for the affected area 12b in the living body 12a can surely be misted. Therefore, the medical solution 11a of the proper amount can be administered to the affected area 12b in the living body 12a. In other words, in the fourth embodiment, the amount of the medical solution 11a necessary for the affected area 12b in the living body 12a can be substantially equalized to the solution sending amount of the medical solution 11a sent by the medical solution sending mechanism 6. That is, the item such as the dose of the remedy which should exactly be managed can be achieved.

In the fourth embodiment, the disposal syringe 21 may have a configuration in which the disposal syringe 21 is not filled with the air 17, similar to that of the first embodiment.

A fifth embodiment of the invention will be described below with reference to FIG. 9. The same components as the first to fourth embodiments are designated by the same numerals, and the descriptions thereof are omitted. FIG. 9 is a schematic view showing the other end of the catheter of the fifth embodiment.

In the fifth embodiment, as shown in FIG. 9, the catheter 31 has plural spray nozzles 31c in the distal-end surface 31d at the other end 31b. Similarly to the third and fourth embodiments, the voltage application thin wire 52 is inserted into the catheter 31. Because other configurations are substantially similar to those of the first to fourth embodiments, the detailed descriptions thereof are omitted.

Therefore, in the fifth embodiment, similarly to the second embodiment, the dose of the medical solution 11a can be increased in the insertion direction of the catheter 31.

In the fifth embodiment, the configuration for increasing the dose of the medical solution 11a is not limited to the configuration of FIG. 9. For example, as shown in FIG. 5, the catheter 31 may have plural spray nozzles 31c in the outer circumferential surface 31e at the other end 31b.

Therefore, the dose of the medical solution 11a can be increased in the radial (circumferential) direction of the catheter 31.

In the fifth embodiment, as shown in FIGS. 5 and 9, because the number of spray nozzles 31c and the layout of the spray nozzles 31c can arbitrarily be set in the catheter 31, the dose of the medical solution 11a can be increased. In the fifth embodiment, similarly to the third embodiment, an electrical flux line 16 is formed between each spray nozzle 31c and the living body 12a, so that the medical-solution fine particulate 11b can be sprayed from each spray nozzle 31c. The increased number of spray nozzles 31c increases the dose, so that the administration of the medical solution 11a of the proper amount can be completed in a short time. In the fifth embodiment, similarly to the third embodiment, the voltage application thin wire 52 is disposed. Therefore, the applied voltage generated by the voltage generation circuit 41 can be lowered even if the dose is increased.

Obviously, a configuration in which the catheter 31 has plural spray nozzles 31c in the distal-end surface 31d or the outer circumferential surface 31e can be applied like the second embodiment. In the fifth embodiment, the dose can be increased even if the voltage application thin wire 52 is not disposed.

The configurations of the first to fifth embodiments may be combined.

(Additional Remark 1)

A liquid spray administration apparatus which is inserted into a body, and sprays and administers a liquid to a desired region in the body to cause the liquid to adhere to the desired region in the body, the liquid spray administration apparatus including:

a liquid filling unit which is filled with the liquid;

a catheter which is connected to the liquid filling unit at one end while having a spray nozzle at the other end, the spray nozzle spraying the liquid;

a voltage generation unit which generates a voltage;

a voltage application unit which is disposed near the liquid filling unit or one end of the catheter, and applies the voltage generated from the voltage generation unit to the liquid; and

a liquid sending mechanism which sends the liquid from the liquid filling unit to the spray nozzle, the voltage being applied to the liquid by the voltage application unit,

wherein an amount of the liquid to which the voltage is applied by the voltage application unit and which is sent from the liquid filling unit to the spray nozzle by the liquid sending mechanism is substantially equalized to an amount of the liquid which is sprayed and administered from the spray nozzle to adhere to the desired region in the body.

(Additional Remark 2)

The liquid spray administration apparatus according to additional remark 1, wherein the desired region in the body is grounded, the liquid which is sprayed and administered from the spray nozzle to adhere to the desired region in the body follows an electrical flux line formed from the spray nozzle toward the desired region in the body, and the liquid is sprayed and administered to the desired region in the body in the form of a charged misty liquid fine particulate.

(Additional Remark 3)

The liquid spray administration apparatus according to additional remark 2, wherein the voltage generation unit controls at least one of polarity of the voltage, magnitude of the voltage, and a duration time in which the voltage is applied to the liquid.

(Additional Remark 4)

The liquid spray administration apparatus according to additional remark 3, wherein the liquid sending mechanism controls at least one of a solution sending speed and a solution sending amount when the liquid is sent from the liquid filling unit to the spray nozzle.

(Additional Remark 5)

The liquid spray administration apparatus according to additional remark 4, wherein a diameter of the particles in the liquid fine particulate can be varied by at least one of the solution sending speed controlled by the liquid sending mechanism and the magnitude of the voltage controlled by the voltage generation unit.

(Additional Remark 6)

The liquid spray administration apparatus according to additional remark 5, wherein the catheter includes plural spray nozzles.

(Additional Remark 7)

The liquid spray administration apparatus according to additional remark 6, wherein the catheter includes plural spray nozzles in an outer circumferential surface.

(Additional Remark 8)

The liquid spray administration apparatus according to additional remark 7, wherein a voltage application thin wire is provided in the voltage application unit, the voltage application thin wire being inserted into the catheter to a neighborhood of the spray nozzle, and applying the voltage to the liquid in the spray nozzle through the liquid near the other end in the catheter.

(Additional Remark 9)

The liquid spray administration apparatus according to additional remark 8, wherein the liquid filling unit, the catheter, and the voltage application unit are detachably attached to the voltage generation unit and the liquid sending mechanism.

(Additional Remark 10)

A liquid spray administration method performed by a liquid spray administration apparatus which is inserted into a body, and sprays and administers a liquid to a desired region in the body to cause the liquid to adhere to the desired region in the body, the liquid spray administration method including:

a step of filling a liquid filling unit with the liquid;

a step of connecting the liquid filling unit and one end of a catheter;

a step of generating a voltage;

a step of applying the voltage to the liquid at the liquid filling unit or one end;

a step of sending the voltage-applied liquid from the liquid filling unit to a spray nozzle provided at the other end of the catheter; and

a step in which an amount of the liquid to which the voltage is applied and which is sent from the liquid filling unit to the spray nozzle is substantially equalized to an amount of the liquid which is sprayed and administered from the spray nozzle to adhere to the desired region in the body.

The invention is not limited to the above-described embodiments, but various modifications can be made without departing from the scope of the invention. Additionally, various inventions can be formed by an appropriate combination of plural constituents disclosed in the embodiments.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A liquid spray administration apparatus which is inserted into a body to spray and administer liquid, comprising:
a liquid filling unit which is filled with the liquid;
a catheter which is connected to the liquid filling unit at one end while having a spray nozzle at the other end, the spray nozzle spraying the liquid;
a voltage generation unit which generates a voltage; and
a voltage application unit which is disposed near the liquid filling unit or one end of the catheter, and applies the voltage generated from the voltage generation unit to the liquid in the spray nozzle through the liquid in the catheter,
wherein the liquid to which the voltage is applied is sprayed and administered in a form of a charged misty liquid fine particulate from the spray nozzle toward a region, the region having a potential which is different from that of the liquid in the spray nozzle.

2. The liquid spray administration apparatus according to claim 1, further comprising a liquid sending mechanism which sends the liquid from the liquid filling unit to the spray nozzle.

3. The liquid spray administration apparatus according to claim 2, wherein a desired region in the body is grounded, and the liquid fine particulate is selectively administered only to the desired region in the body while following an electrical flux line formed from the spray nozzle toward the desired region in the body.

4. The liquid spray administration apparatus according to claim 3, wherein the voltage generation unit controls at least one of the polarity of the voltage, magnitude of the voltage, and a duration time in which the voltage is applied to the liquid.

5. The liquid spray administration apparatus according to claim 4, wherein the liquid sending mechanism controls at least one of a solution sending speed and a solution sending amount when the liquid is sent from the liquid filling unit to the spray nozzle.

6. The liquid spray administration apparatus according to claim 5, wherein a diameter of the particles of the liquid fine particulate can be varied by at least one of the solution sending speed controlled by the liquid sending mechanism and the magnitude of the voltage controlled by the voltage generation unit.

7. The liquid spray administration apparatus according to claim 6, wherein the voltage application unit is disposed outside the body.

8. The liquid spray administration apparatus according to claim 7, wherein the catheter includes a plurality of the spray nozzles.

9. The liquid spray administration apparatus according to claim 8, wherein the catheter includes the spray nozzle in an outer circumferential surface.

10. The liquid spray administration apparatus according to claim 9, wherein a voltage application thin wire is provided in the voltage application unit, the voltage application thin wire being inserted into the catheter to a neighborhood of the spray nozzle and applying the voltage to the liquid in the spray nozzle through the liquid near the other end in the catheter.

11. The liquid spray administration apparatus according to claim 2, wherein the liquid filling unit, the catheter, and the voltage application unit are detachably attached to the voltage generation unit and the liquid sending mechanism.

12. A liquid spray administration apparatus which is inserted into a body to spray and administer a liquid fine particulate, comprising:
a production unit which produces the charged fine particles from the liquid to which a voltage is applied; and
a spray nozzle which sprays the charged fine particles to a desired region in the body,
wherein the fine particles are sprayed and administered from the spray nozzle toward a region, the region having a potential which is different from that of the liquid in the spray nozzle.

13. The liquid spray administration apparatus according to claim 12, wherein the production unit includes:
a liquid filling unit which is filled with the liquid;
a catheter which is connected to the liquid filling unit at one end;
a voltage generation unit which generates a voltage; and
a voltage application unit which is disposed near the liquid filling unit or one end of the catheter, and applies the voltage generated from the voltage generation unit to the liquid in the spray nozzle through the liquid in the catheter,
wherein the spray nozzle is disposed at the other end of the catheter.

14. A liquid spray administration method performed by a liquid spray administration apparatus which is inserted into a body to spray and administer a liquid, the method comprising:
a step of filling a liquid filling unit with the liquid;
a step of connecting the liquid filling unit and one end of a catheter;
a step of generating a voltage;
a step of applying the voltage to the liquid at the liquid filling unit or one end; and
a step of spraying and administering the liquid to which the voltage is applied in a form of a charged misty liquid fine particulate from the spray nozzle toward a region, the spray nozzle being provided at the other end of the catheter, the region having a potential which is different from that of the liquid in the spray nozzle.
